(19) United States
(12) Patent Application Publication
(10) Pub. No.: US 2010/0057170 A1
(43) Pub. Date: Mar. 4, 2010

(54) Full view access upper underbody blanket

(75) Inventors: John Robinson, Imperial Beach, CA (US); Alan Stec, East Bridgewater, MA (US)

Correspondence Address: LOUIS WOO;LAW OFFICE OF LOUIS WOO, 717 NORTH FAYETTE STREET, ALEXANDRIA, VA, 22314, US

(73) Assignee: Smiths Medical ASD, Inc., Rockland, MA (US)

(21) Appl. No.: 12/230,777

(22) Filed: Sep. 4, 2008

(51) Int. Cl. A61F 7/00

(52) U.S. Cl. 607/107

(57) ABSTRACT

The inventive blanket is an inflatable underbody convective thermal blanket whereon the head and the upper body of a patient may lie. The blanket is in the shape of a cross with a truncated lower vertical portion and an upper head portion onto which the head of the patient lies. The blanket has two horizontal arm portions that extend from the main body portion of the blanket and onto which the arms of the patient may be positioned. At each of the arm portions there is a flap that may be used to cover the arm of the patient. An inlet port is provided in an upper corner at the head portion of the blanket. The heated air input to the blanket is directed by a plurality of sealed strips to flow in respective desired directions around the arm portions and the lower truncated body portion of the blanket. Sets of apertures are provided along an inner sealed periphery that defines a T-shaped uninflated area onto which the torso of the patient lies. There are more apertures at the inner corners of the inflatable structure defined by the uninflated area to enable a greater outflow of heated air at the lower truncated body portion of the blanket. All inner and outer corners of the blanket are rounded to facilitate the air flow.

### FIELD OF THE INVENTION

The present invention relates to convective warming blankets and more particularly to an underbody blanket for warming the upper torso or upper body of a patient.

### BACKGROUND OF THE INVENTION

There are currently upper body blankets that are used to warm the arms and upper body of a patient. One such blanket is disclosed in related U.S. Pat. Nos. 5,360,439, 5,384,924, 5,514,169 and 5,839,133. The blanket disclosed in the noted patents is placed over a patient, and has a recess portion for the head of the patient, so that the head of the patient is not covered by the blanket. Inlet ports are provided at the blanket at either side of the head of the patient. As far as can be ascertained from the disclosure of the noted patents, as the blanket is placed over the patient, heated air is mainly directed to the covered upper body of the patient, with the head of the patient exposed.

There is also disclosed an underbody blanket in U.S. Pat. Nos. 6,102,936 and 6,511,501. The blanket disclosed in those patents is a pediatric blankets that is to be placed underneath an infant patient, so that the entire body of the infant is exposed. There is a head portion whereupon the head of the patient lies. No heat is provided to the head of the patient, as all of the holes whereby heated air outputs from the blanket are located in the main body portion of the blanket.

### SUMMARY OF THE PRESENT INVENTION

The blanket of the present invention is an underbody thermal convective blanket for the upper body or torso of the patient. The blanket is in the shape of a cross with a truncated vertical lower portion and a vertical upper portion onto which the head of the patient lies. Extending from the body portion of the blanket are two horizontal arm portions whereupon the arms of the patient rest. At each of the arm portions there is a transparent flap that may be moved to cover the arm of the patient. A T-shaped uninflated or non-inflated area is provided in the body and armed portions of the blanket whereupon the upper torso and arms of the patient may be positioned. A circular uninflated or non-inflated area in the head portion likewise provides a place onto which the head of the patient may be positioned.

To inflate the blanket, an inlet port is provided at an upper corner of the head portion of the blanket. Parallel rows of air holes or apertures are provided in the head portion for surrounding a major portion of the uninflated area at the head portion. Air apertures are also provided at the head portion, as well as the periphery of the inflated portion of the blanket that, except for the distal ends of the arm portions, surrounds the T-shaped uninflated area. Thus, air apertures are provided at the inflatable portion of the blanket both above and under the arms of the patient. Additional air apertures are provided at the junctions where the arm portions and the lower part of the body portion meet for outputting additional heated air to the patient. A number of sealing strips are formed at the head portion of the blanket for guiding the heated air input to the inlet port along the head portion as well as along and around the arm and body portions of the blanket.

To maintain warmth to the arms of the patient, a flexible flap, which may be transparent, clear or opaque, is attached to each of the arm portions. Optionally, fluid absorbent pads may be attachedly provided at the T-shaped uninflated area of the blanket for absorbing liquid that may be collected on the blanket. To enhance the flow of air along the blanket, all corners, both external and internal, of the blanket are rounded.

The present invention therefore relates to an underbody blanket for supporting the upper body or torso of a patient. The underbody blanket comprises an inflatable structure having a head portion, a body portion and two arm portions. The head portion is connected to the body portion, a corresponding one of the arm portions extending from each side of the body portion. The body portion has a non-inflatable area that extends into the arm portions for supporting the upper torso of the patient. The head portion has an uninflated area for supporting the head of the patient. Respective sets of apertures are formed along sections of the inflatable structure that bound the uninflated area of the head and body portions.

The present invention further relates to an underbody blanket for supporting the upper body of the patient that comprises an inflatable structure having a head portion for supporting the head of a patient, a body portion for supporting the upper torso of the patient, and two arm portions extending from a corresponding side of the body portion for supporting the arms of the patient. The blanket further comprises a substantially T-shaped uninflated area in the body portion bounded by the head portion that extends into each of the arm portions whereon the upper torso and the arms of the patient are positionable. Sets of apertures are provided along respective sections of the inflatable structure. The inflatable structure defines two lower corners of the T-shaped uninflated area where the body and arm portions meet. A greater number of apertures are provided at those sections of the inflatable structure that define the two lower corners than at other sections of the inflatable structure.

The present invention further relates to a method of making an underbody convective blanket for warming the upper body of the patient lying thereon. The method comprises the steps of: attaching air impermeable upper and lower layers to form a sealed inflatable structure having a head portion, a body portion and two arm portions each extending from a corresponding side of the body portion; forming a substantially T-shaped uninflatable area bounded by the head portion and the arm portions whereupon the upper torso of the patient is positionable; forming apertures in the layer of the structure that comes into contact with the torso of the patient along respective sections of the inflatable structure that define the body and the arm portions, the inflatable structure defining two lower corners of the T-shaped uninflated area where the body and the arm portions meet; and providing a greater number of apertures at those sections of the inflatable structure that define the two lower corners than at other sections of the inflatable structure.

### BRIEF DESCRIPTION OF THE FIGURES

The present invention will become apparent and will best be understood by reference to the following description of the invention taken in conjunction with the accompanying drawings, wherein:

FIG. 1 is a plan view of the upper body underbody blanket of the present invention;

FIG. 2 is a cross-sectional view of the blanket along view 2-2 as shown in FIG. 1; and

FIG. 3 is another view of the blanket of the present invention having superposed thereon in phantom line the body of a patient.

### DETAILED DESCRIPTION OF THE INVENTION

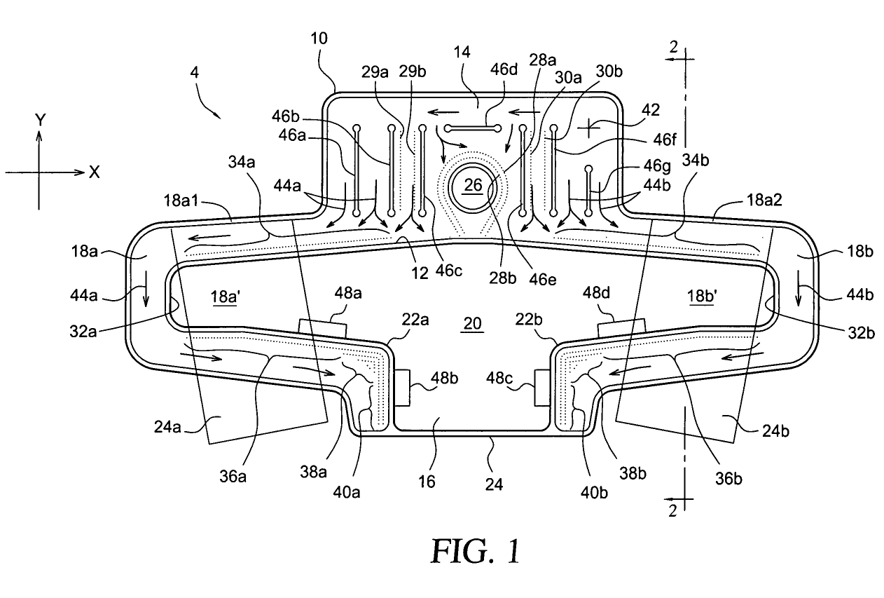

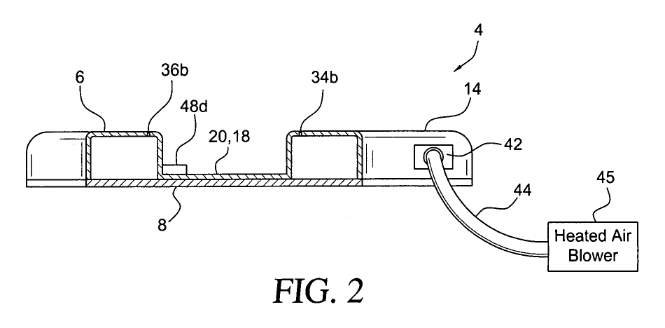

With reference to FIGS. 1 and 2, the present invention convective warming blanket 4 is an inflatable structure made up of an upper air impermeable layer 6 which makes contact with a patient and a lower air impermeable layer 8. The upper and lower layers 6 and 8 are bonded together by a conventional sealing process at different areas of the blanket, as for example at the sealed outer periphery 10 and the sealed inner periphery 12. As shown, the overall blanket 4 is in the shape of a cross, with the lower vertical portion of the cross truncated.

As best shown in FIG. 1, blanket 4 includes a head portion 14, a body portion 16 and arm portions 18a and 18b, each of which extends from a corresponding side of body portion 16. The inflatable portion of the blanket may be identified by that area or portion sandwiched between the outer seal periphery 10 and the inner seal periphery 12. Thus, the blanket has an uninflated T-shaped area 20 at body portion 16 bounded by arm portions 18a and 18b, and a lower sealed edge 24. Arm portions 18a and 18b each are inflatable to be tube shaped. Area 20 includes the uninflated arm areas 18a′ and 18b′ each surrounded at three sides by the inflatable tubular arm portions 18a and 18b, respectively.

As further shown in FIG. 1, the uninflated arm areas 18a′ and 18b′ each are an extension of uninflated area 20 of the body portion 16 so that at the junction where the uninflated areas of the body and arm portions meet, two inner corners 22a and 22b are formed or defined by the inflatable portion of the blanket that further extends to the lowermost edge 24 of the blanket, which also may be referred to as the lowermost edge of area 20. These lower inflatable extensions of arm portions 18a and 18b may be referred to as the legs of the inflatable portion. At edge 24, the outer and inner sealed peripheries 10 and 12 are merged to form the lower boundary of the body portion for the blanket. To enhance the flow of air along the various portions of the blanket, all corners, both external and internal, of the blanket are rounded. See for example corners 22a and 22b.

Two flexible flaps 24a and 24b are connected to arm portions 18a and 18b at their respective upper edges 18a1 and 18a2. These flexible flaps may be made of transparent, clear or opaque PVC or other flexible materials and are movable between a position away from areas 18a1 and 18a2 to the positions as shown in FIG. 1 for covering the arms of a patient lying on blanket 4.

Blanket 4 further has a circular uninflated area 26 at its head portion 14 whereby the head of the patient is positionable. A double row of apertures 28a and 28b are provided at the head portion to surround the major portion of circular uninflated area 26. Additional rows of apertures 29a, 29b and 30a, 30b are provided longitudinally, along the y direction as shown in FIG. 1, at the head portion 14 of the blanket.

With respect to the body portion 16 and the arm portions 18a and 18b, except at the distal ends 32a and 32b of the arm portions 18a and 18b, there are a plurality of rows of air apertures provided along different sections on the inflatable upper layer 6 adjacent the inner periphery 12. As shown, respective rows of apertures 34a and 34b are provided at the inflatable portion adjacent to the inner periphery 12 that defines the uninflated T-shaped area 20. Note that the spacing of the apertures at rows 34a and 34b increases toward the center of the blanket but remains constant at the arm portions 18a and 18b.

Another set of respective rows of apertures 36a and 36b are provided to those sections of the inflatable portion adjacent to the inner periphery 12 that define the lower sections of inflatable arm portions 18a and 18b. The apertures are evenly spaced for the rows of apertures 36a and 26b. There are however a greater number of apertures provided at the cornered portions of the inflatable structure that define inner corners 22a and 22b of the uninflated area 20. These sets of apertures are designated 38a and 38b, respectively, in FIG. 1. A last dual set of apertures, each of which comprises a double row of apertures 40a and 40b, substantially extend from the inner corners 22a and 22b along the lower legs of the inflatable portion to the lower edge 24 of the blanket. Thus configured, air flow is promoted throughout the blanket, and a relatively constant temperature gradient is maintained for the air being circulated, even for the lower legs of the inflatable portion adjacent to body portion 16.

The temperature treated air, for example heated air, is input to the blanket 4 at inlet port 42, via for example an air hose 44 mated to the inlet port 42. The other end of air hose 44 in turn is connected to a heated air blower 45, for example an EQUATOR unit sold by the assignee of the present invention, so that heated air may be input to blanket 4 for inflating the blanket.

To enhance the circulation of air, and in particular directing the flow of air from inlet port 42 through head portion 14 and then via the paths as indicated by the directional arrows 44a and 44b to the remainder of the blanket, a number of sealing strips, for example 46a-46g, are provided at the head portion 14 of the blanket. The sealing strips 46a-46g are in spatial relationship to each other and are positioned such that the flow of air input from inlet port 42 may be directed, per paths 44a and 44b, to both arm portions 18a and 18b, and eventually from there to the lower legs of the inflatable portion that define the lower section of uninflated area 20 of body portion 16. With an increased number of apertures at inner corners 22a and 22b, per designated by aperture sections 38a and 38b, and the double rows of apertures 40a and 40b at the lower portion of body portion 16, more air is output at those portions of the blanket that are most remote from inlet port 42. As a result, the different portions of the upper torso or body of the patient lying on the blanket would be warmed substantially evenly, due to a greater amount of heated air being output from those portions of the blanket that are remote from the air inlet.

There are no apertures at distal ends 32a and 32b of the arm portions 18a and 18b. This is to ensure that those areas of the blanket onto which the hands and lower arms of the patient are to be positioned should not be heated, for it is highly likely that IV lines or other tubes and devices may need to be connected to those limbs of the patient. And these lines and devices, as well as the fluid in the IV lines, may be affected by heat. Thus, the hand areas of the blanket are designed not to have heated air output thereat.

Returning to the sealing strips, for directing the air flow in the blanket, strips 46g and 46f would direct the input air toward arm portion 18b. Moreover, with the sealing strip 46d configured in the x direction as shown, air flow is directed toward the area of head portion 14 away from air inlet port 42, so that the heated air may also be directed to the area of head portion 14 that contains strips 46a-46c, which may then further direct the heated air along paths 44a to arm portion 18a. Heated air also circulates about the uninflated area 26 at head portion 14, so that heated air is output from the rows of apertures 28a and 28b for warming the head of the patient.

Further shown with respect to FIGS. 1 and 2 are optional fluid absorption pads 48a-48d that may be attached to different areas of the T-shaped uninflated area 20, for absorbing liquid that may be collected thereat, from either the patient or the medical personnel using it on the patient.

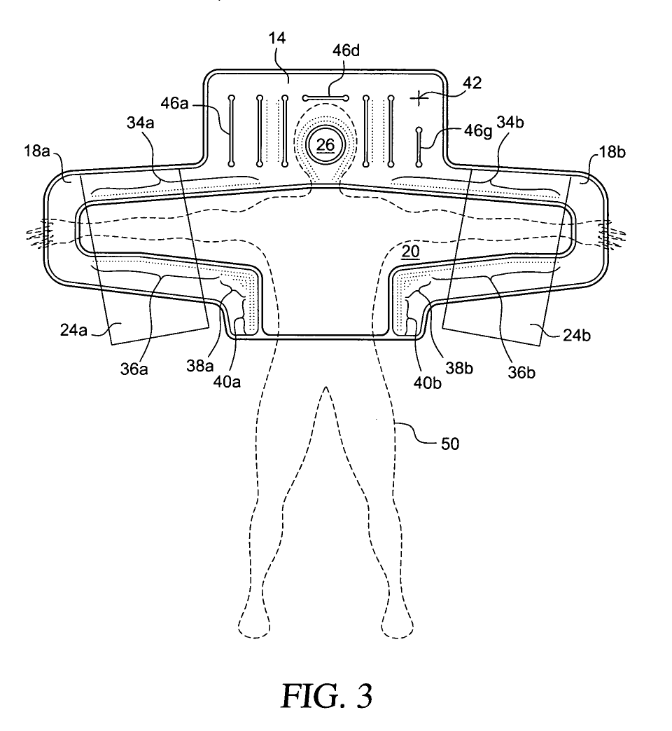

FIG. 3 shows the blanket of the present invention having positioned thereon an exemplar patient 50, shown in phantom line. As shown, the head of the patient rests on uninflated area 26 while his upper torso is positioned substantially on the uninflated area 20, with his arms extending and resting along arm areas 18a′ and 18b′. The upper torso and arms of the patient accordingly are warmed by the heated air output from the respective sets of air outlet apertures 34a and 34b, 36a and 36b, 38a and 38b, and 40a and 40b provided at the inflatable upper layer 6 along the inner sealed periphery 12 that defines the uninflated area 20. Also, with the patient resting on the blanket, the head and upper torso of the patient are fully accessible to the medical personnel. To maintain the heat on the arms of the patient, flaps 24a and 24b are shown in FIG. 3 to have been moved to cover the arms of the patient.

The invention disclosed above is subject to various variations, modifications and changes in detail. Accordingly, it is intended that all matters described throughout this specification and shown in the accompanying drawings be interpreted as illustrative only and not in a limiting sense. It is therefore intended that the invention be limited only by the spirit and scope of the hereto appended claims.

1. An underbody blanket for supporting the upper body of a patient, comprising: an inflatable structure having a head portion, a body portion, and two arm portions, said head portion being connected to said body portion, a corresponding one of said arm portions extending from each side of said body portion, said body portion having a non-inflatable area that extends into said arm portions for supporting the upper torso and arms of the patient, said head portion having an uninflated area for supporting the head of the patient, respective sets of apertures formed along sections of the inflatable structure that bound the uninflated area of said body portion.

2. Blanket of claim 1, further comprising: an inlet at said head portion to enable temperature treated air to be input to the inflatable structure; and seal strips provided in relative spatial relationship to each other in said head portion to guide the flow of air from said head portion to said body and arm portions.

3. Blanket of claim 1, wherein at least all internal corners of the uninflated areas of said blanket are rounded.

4. Blanket of claim 1, wherein the uninflated area of said body portion extends to the lowermost edge of said body portion such that said arm portions of said blanket each comprise a tube surrounding the uninflated area.

5. Blanket of claim 1, wherein additional apertures are provided at respective sections of the inflatable portion that bound corresponding corners of the uninflated area that join said body portion and said arm portions, more apertures provided along said respective sections and sections of the inflatable portion along said body portion that extends from the corners than those sections of the inflatable portion that bound the uninflated area along said arm portions.

6. Blanket of claim 1, further comprising two flaps each at one of said arm portions of the blanket, each of said flaps movable to cover a corresponding arm of the patient lying on said blanket.

7. Blanket of claim 1, further comprising at least one liquid absorbent pad at the uninflated area of said body and arm portions for absorbing liquid collected on said uninflated area.

8. An underbody blanket for supporting the upper body of a patient comprising an inflatable structure having a head portion for supporting the head of the patient, a body portion for supporting the upper torso of the patient, and two arm portions each extending from a corresponding side of said body portion for supporting the arms of the patient, a substantially T shaped uninflated area in said body portion bounded by said head portion and extending along a portion of each of said arm portions whereon the upper torso and the arms of the patient are positionable, sets of apertures provided along respective sections of the inflatable structure, the inflatable structure defining two lower corners of the T shaped uninflated area where said body and arm portions meet, a greater number of apertures provided at those sections of the inflatable structure that define said two lower corners than at other sections of the inflatable structure.

9. Blanket of claim 8, further comprising a substantially circular uninflated area at said head portion where the head of the patient is positionable, a plurality of apertures surrounding at least a portion of said circular uninflated area.

10. Blanket of claim 8, further comprising an inlet provided at said head portion to enable temperature treated air to be input to said structure, the input air traversing from said head portion to said body portion by flowing about the T shaped uninflated area.

11. Blanket of claim 10, further comprising a plurality of sealing strips at said head portion for directing the flow of input air to said arm and body portions of said blanket.

12. Blanket of claim 8, wherein the T shaped uninflated area extends to the lowermost edge of said body portion such that said arm portions of said blanket each comprise an inflatable tube surrounding an uninflated area.

13. Blanket of claim 8, further comprising two flaps each at one of said arm portions of the blanket, each of said flaps movable to cover a corresponding arm of the patient lying on said blanket.

14. Blanket of claim 8, further comprising at least one liquid absorbent pad at the T shaped uninflated area for absorbing liquid collected on the uninflated area.

15. Blanket of claim 8, wherein all corners of said blanket are rounded.

16. A method of making an underbody convective blanket for warming the upper body of a patient lying thereon, comprising the steps of:
attaching air impermeable upper and lower layers to form a sealed inflatable structure having a head portion, a body portion and two arm portions each extending from a corresponding side of said body portion;
forming a substantially T shaped uninflated area bounded by at least said head portion and said arm portions whereupon the upper torso of the patient is positionable;
forming apertures in the layer of said structure that comes into contact with the torso of the patient along respective sections of the inflatable structure that define said body and arm portions, the inflatable structure defining two lower corners of the T shaped uninflated area where said body and arm portions meet; and
providing a greater number of apertures at those sections of the inflatable structure that define said two lower corners than at other sections of the inflatable structure.

17. Method of claim 16, further comprising the step of forming respective sets of apertures at corresponding inflatable portions above and below the uninflated areas of said arm portions.

18. Method of claim 16, further comprising the steps of:
forming a substantially circular uninflated area at the head portion where the head of the patient is positionable; and
providing a plurality of apertures about at least a portion of the circular uninflated area to output heat to the head of the patient.

19. Method of claim 16, further comprising the steps of:
providing an inlet at said head portion to enable temperature treated air to be input to said structure; and
directing the flow of air from said head portion to said body portion by routing the air flow about the T shaped uninflated area.

20. Method of claim 16, further comprising the step of sealing said head portion with a plurality of strips at spatial relationship to each other for directing the flow of input air to the inflatable arm and body portions of said blanket.

21. Method of claim 16, further comprising the step of attaching two flaps each at one of said arm portions of the blanket, each of said flaps movable to cover a corresponding arm of the patient lying on said blanket.

22. Method of claim 16, further comprising the step of rounding all corners of said blanket.
